(19) United States
(12) Patent Application Publication
(10) Pub. No.: US 2010/0222858 A1
(43) Pub. Date: Sep. 2, 2010

(54) METHOD AND SYSTEM FOR NEURALLY AUGMENTING SEXUAL FUNCTION DURING SEXUAL ACTIVITY

(76) Inventor: T. Stuart Meloy, Mocksville, NC (US)

Correspondence Address: WOMBLE CARLYLE SANDRIDGE & RICE, PLLC, ATTN: PATENT DOCKETING, P.O. BOX 7037, ATLANTA, GA, 30357-0037, US

(21) Appl. No.: 12/394,230

(22) Filed: Feb. 27, 2009

(51) Int. Cl. A61N 1/00; A61F 5/00

(52) U.S. Cl. 607/117; 600/38; 607/72

(57) ABSTRACT

A method and system for neurally augmenting the sexual function during sexual activity between two participants. A sensor is implanted in the spinal cord of each participant and connected to a current generator, receiver and transmitter. Neural impulses occurring in the spinal cord are transmitted back and forth between the participants, so that one or both parties are sensing the neural sensations occurring in the other.

### FIELD OF THE INVENTION

The invention generally relates to communication of neural activity between participants during a sexual experience, and more particularly relates to a method and system for permitting one person to physically sense neural sensations being experienced by a partner during sexual activity, and when desired, to artificially augment a person's sexual function during such sexual activity.

### BACKGROUND

Electrical spinal cord stimulation is known for treating persons with chronic pain. Such a procedure typically involves inserting electrodes into the epidural space of a person's spinal column, and applying electrical impulses to the spinal cord with the electrode. Implantable pulse generators (IPGs) are known for use in such treatment. A typical IPG generally includes a battery-powered current generator connected by wires or leads to one or more electrodes placed within a selected portion of a person's spinal cord. An internal controller within the IPG controls the transmission of electrical impulses to the electrodes, including the frequencies and amplitudes of the transmitted electrical impulses. The IPG also can include an electronic communication device for wirelessly communicating data and control signals between the IPG and an external device, such as an external controller. The IPG can also include a memory which permits the IPG to be programmed to generate a particular pattern, series or sequence of electrical impulses. As used herein the terms pattern of electrical impulses, series of electrical impulses or sequence of electrical impulses may refer to a single electrical impulse having a particular amplitude and duration, or a plurality of sequential electrical impulses having a particular frequency or particular frequencies, a particular amplitude or particular amplitudes, and a particular duration or particular durations. Typical IPGs are sufficiently small to be implanted subcutaneously in a person. The batteries of an implanted IPG can be recharged wirelessly via radio frequency energy produced by a nearby radio frequency transmitter and received by charging circuitry within the IPGs. Such IPGs and neuro-stimulation systems are commercially available from Medtronics, St. Judes and Boston Scientific, for example.

As described in U.S. Pat. No. 6,169,924 to Meloy et al., electrical spinal cord stimulation can also be used to produce sexual arousal and an orgasmic response in a person. Stimulating electrodes can be placed in the epidural space within a selected portion of the spine and in parallel with the spinal cord. As described in U.S. Pat. No. 6,169,924, preferred selected areas of the spine include T8 to S3, inclusive of the lumbar, and more preferably, T11 to L2. Other sites along the spinal cord are also possible. The electrodes can be coupled to a power source. By providing electrical impulses to the selected portion of a person's spinal cord via the power source and electrodes, a person can be sexually aroused to the point of orgasm. Such a procedure can be used to treat persons with orgasmic dysfunction, for example. An IPG like that described above can be used to generate and deliver the desired electrical impulses to the implanted electrodes. The disclosure of U.S. Pat. No. 6,169,924 is hereby incorporated by reference in its entirety.

### SUMMARY

Sexual activity between two persons can be more satisfying and fulfilling when both persons achieve a similarly high degree of physical and emotional arousal and pleasure. In particular, sexual partners may attain greater satisfaction and fulfillment when both partners achieve orgasm during sexual intercourse or another sexual activity. Even more preferably, sexual partners may prefer that both or all partners achieve orgasm substantially simultaneously during a sexual activity. As used herein, the terms sexual activity, sexual act and similar terms refer to any activity involving two or more persons which is capable of sexually arousing one or more persons involved in the sexual activity or act, including without limitation: sexual intercourse, cunnilingus, fellatio, fondling, intimate touching, observing an erotic act or performance, viewing a sexually explicit film or photograph, listening to erotic speech or sounds, conduct associated with a person's fetish, and any other form of sexually arousing activity or event.

In some instances, one sexual partner may achieve a higher degree of sexual arousal and fulfillment than another sexual partner achieves during a particular sexual activity. For example, only one sexual partner may achieve orgasm during a sexual activity. Because not all persons respond the same way to a particular type or style of sexual stimulation or inducement, a person may not know how to best physically or otherwise sexually arouse a particular sex partner. For example, a person may not understand how to physically stimulate a particular partner's genitalia or other sensitive body part in a way that will produce a high degree of sexual arousal or orgasm. In addition, a person may not know which particular portion of a partner's genitalia or another body part is most likely to provide pleasure to the partner when properly stimulated or manipulated.

Because a person cannot physically feel the neural sensations being experienced by a partner during a sexual act, the person may not know when or how to adjust their own sexual performance or behavior in a way that will increase or maximize the degree or quality of sensual pleasure being experienced by their partner. Conversely, a person may not realize that they are arousing their sexual partner too intensely or too quickly, and that they may need to adjust their performance or behavior in order to prolong sexual activity. In addition, a person may be unsure how a specific change in their own performance or behavior actually affects their partner's sensual response to the altered performance or behavior.

Accordingly, it would be beneficial for a person to be able to physically sense the sensations being experienced by a sex partner during sexual activity such that the person can better understand when or how to adjust their behavior or performance in a way that will increase or decrease the degree of sensual arousal being experienced by their partner. In particular, it would be useful for a person to be able to physically sense a partner's sensual response in real time during a sexual activity. In addition, it would be beneficial to be able to artificially augment a person's sexual function or state of sexual arousal during a sexual activity based on a sex partner's arousal response to the sexual activity.

### BRIEF DESCRIPTION OF THE DRAWINGS

FIG. 1 is a schematic diagram showing one embodiment of a system and method for neurally augmenting sexual function in a person;

FIG. 2 is a schematic diagram showing another embodiment of a system and method for neurally augmenting sexual function in one person or two persons;

FIG. 3 shows a neuromodulation device arranged for use in the systems and methods depicted in FIGS. 1 and 2.

FIG. 4 is another schematic diagram illustrating details of the system and method shown in FIG. 2.

FIG. 5 is a schematic diagram showing another embodiment of a system and method for neurally augmenting sexual function in one or more persons in a group of more than two people.

### DETAILED DESCRIPTION

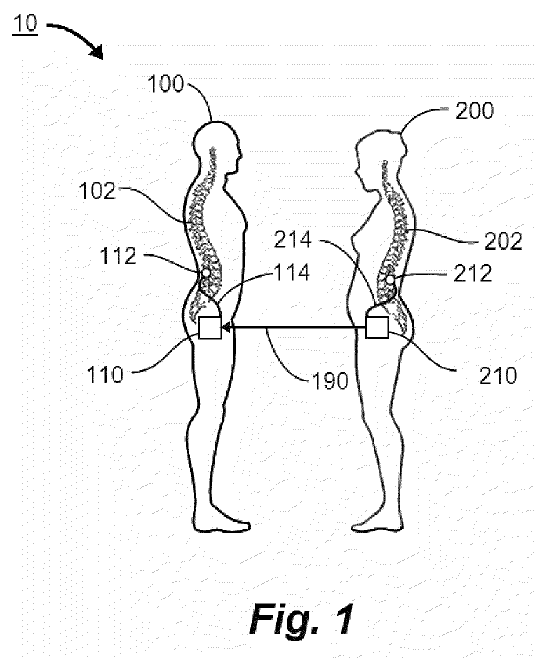

FIG. 1 illustrates one embodiment of a neural augmentation system 10 for enhancing or supplementing the sexual function or response of a first person 100 while that person is engaged in sexual activity with a second person 200. As shown schematically in FIG. 1, a first electrode or sensor 112 can be implanted in a selected portion of the spine 102 of a first person 100, and the first electrode 112 can be coupled to a first neuromodulation device 110 by a wire or lead 114. Similarly, a second electrode 212 can be implanted in a selected portion of the spine 202 of a second person 200, and the second electrode 212 can be coupled to a second neuromodulation device 210 by a wire or lead 214. The second neuromodulation device 210 can be adapted to detect a first pattern of electrical neural impulses in the selected portion of the second person's spine 202 via the second electrode 212 and the lead 214 during sexual activity between the first person 100 and second person 200. The second neuromodulation device 210 can also be adapted to communicate characteristics of the first pattern of electrical neural impulses to the first neuromodulation device 110 via a first communication link 190. Preferably, the first and second neuromodulation devices 110, 210 are implanted subcutaneously, for example, in the first and second persons 100, 200, respectively.

The first neuromodulation device 110 can be configured to receive the communicated electrical impulse characteristics from the second neuromodulation device 210, and to respond by generating a second pattern of electrical impulses. The generated second pattern of electrical impulses can be transmitted from the first neuromodulation device 110 to the selected portion of the first person's spine 102 via the first lead 114 and first electrode 112. The second pattern of electrical impulses can be substantially identical to the first pattern of electrical neural impulses, or can include one or more modifications to the first pattern of electrical impulses. In one embodiment, the second pattern of electrical impulses substantially duplicates or mimics the first pattern of electrical neural impulses, and is substantially contemporaneous with the first pattern of electrical neural impulses. When the second pattern of electrical impulses is applied to the first person's spine 102, the first person 100 may physically sense, feel or experience sensations which substantially duplicate or mimic at least some physical sensations being experienced by the second person 200. In particular, when the first pattern of electrical neural impulses is associated with neural activity related to sexual arousal of the second person 200, the first person 100 may experience a substantially similar state of sexual arousal. Accordingly, the system 10 can be used to augment the sexual function of the first person 100 by applying a pattern of electrical impulses to the first person's spine 102 which substantially duplicates or mimics a pattern of electrical neural activity being contemporaneously experienced by the second person 200.

Because the first person 100 can sense the degree of neural excitation and associated sexual arousal in the second person during the sexual activity, the first person 100 can determine when and whether to modify his or her own performance or behavior to maintain or alter the arousal response of the second person. For example, the first person 100 can sense whether their partner 200 is or is not being sexually aroused by the first person's present actions or behavior, and accordingly, can determine whether to continue or discontinue a particular action or behavior.

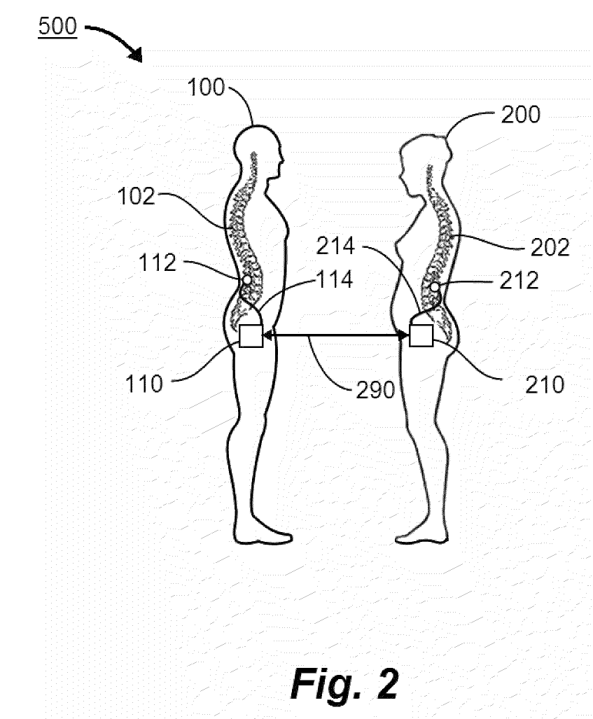

In the neural augmentation system 10 and method depicted in FIG. 1, the characteristics of the first pattern of electrical impulses are unidirectionally communicated from the second neuromodulation device 210 to the first neuromodulation device 110. In the system 500 and method depicted in FIG. 2, however, both the first neuromodulation device 110 and the second neuromodulation device 210 are adapted to transmit information to the other device 110, 210, and to receive information from the other device 110, 210. As shown in FIG. 2, the first neuromodulation device 110 can be adapted to detect a third pattern of electrical neural impulses in the selected portion of the first person's spine 102 via the first electrode 112 and the lead 114 during sexual activity with the second person 200. The first neuromodulation device 110 can also be adapted to communicate characteristics of the third pattern of electrical neural impulses to the second neuromodulation device 210 via a parallel communication link 290.

The second neuromodulation device 210 can be configured to receive the communicated electrical impulse characteristics from the first neuromodulation device 110, and to respond by generating a fourth pattern of electrical impulses. The generated fourth pattern of electrical impulses can be transmitted from the second neuromodulation device 210 to the selected portion of the second person's spine 202 via the second lead 214 and second electrode 212. The generated fourth pattern of electrical impulses can be substantially identical to the detected third pattern of electrical neural impulses, or can include one or more modifications to the third pattern of electrical impulses. In one embodiment, the fourth pattern of electrical impulses substantially duplicates the third pattern of electrical neural impulses, and is substantially contemporaneous with the third pattern of electrical neural impulses. When the fourth pattern of electrical impulses is applied to the second person's spine 202, the second person 200 may sense or feel sensations which substantially duplicate or mimic nervous sensations being experienced by the first person 100. In particular, when the third pattern of electrical neural impulses is associated with sexual arousal of the first person 100, the second person 200 may experience a substantially similar state of sexual arousal. Accordingly, the system 300 can be used to substantially simultaneously augment the sexual function of both the first person 100 and the second person 200 by applying a pattern of electrical impulses to each person's spine 102, 202 which substantially duplicates a pattern of electrical neural activity being contemporaneously experienced by the other person 100, 200. Because both the first person 100 and second person can sense the degree of sexual arousal being experienced by the other during sexual activity, each person 100, 200 can separately determine when and whether to modify his or her own performance to alter the arousal response of the other person.

As will become evident, the communication of neural activity between the two persons 100, 200 can enhance the sexual experience to new heights previously unattainable. This technique can be used by a person who experiences atypically low levels of sexual responsiveness during sexual activity, such as person with orgasmic dysfunction, for example. Alternatively, the technique can be used by persons with typical or high levels of sexual responsiveness in order to enhance their sexual experience, for example.

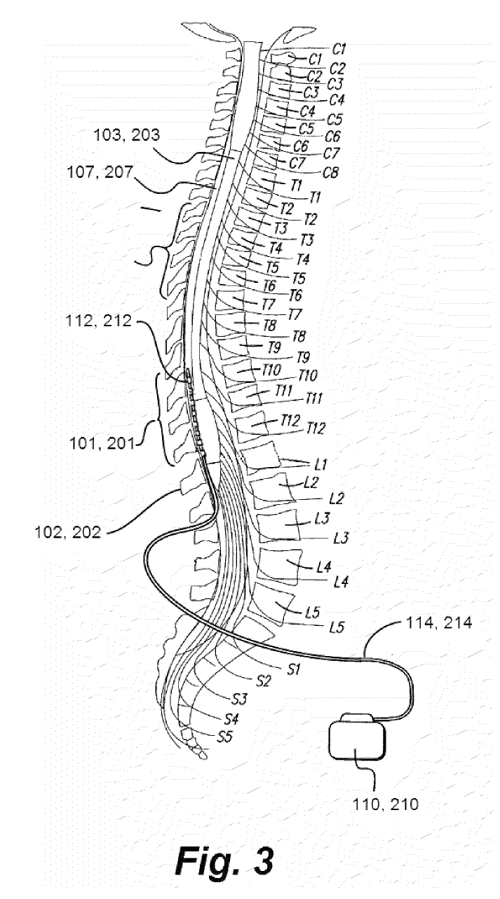

One type of device for use as the first neuromodulation device 110 and/or the second neuromodulation device 210 described above is shown in FIG. 3. In this embodiment, the neuromodulation device 110, 210 is a commercially available implantable pulse generator (IPG). As discussed above, IPGs are implantable devices that can be used to generate a desired series of electrical impulses and to apply the series of electrical impulses to a person's spinal cord for the purpose of pain therapy, or the like. Such an IPG 110, 210 can be electrically coupled to an electrode 112, 212 by a lead 114, 214. The electrode 112, 212 can be a single electrode, or can be an array of electrodes. As shown in FIG. 3, the electrode 112, 212 can be inserted into the epidural space 107, 207 of a person's spinal column 102, 202 such that the electrode 112, 212 is adjacent to a selected portion 101, 201 of the person's spinal cord 103, 203. The portion of the spinal cord 103, 203 selected for placement of the electrode 112, 212 can be a region of the spinal cord 103, 203 known to be a locus for electrical neural activity associated with sexual arousal, for example. In one embodiment, the electrode 112, 212 is preferably located between vertebra T8 and vertebra S3, inclusive of the lumbar region, and more preferably, is located between vertebra T11 and vertebra L2. It is however, possible that in some patients, the electrodes can be located in the other areas of the spinal cord in which neural activity associated with sexual activity has been noted.

Each IPG 110, 210 can be adapted to generate a desired pattern of electrical impulses to be applied to a portion of a person's spinal cord 103, 203 via its associated lead 114, 214 and electrode 112, 212. Each IPG 110, 210 can also be adapted to detect a pattern of electrical nerve impulses in a person's spinal cord 103, 203 via its associated lead 114, 214 and electrode 112, 212. For example, one or both of the of the IPGs 110, 210 could be adapted to sense a series or pattern of electrical neural impulses in a person's spinal cord resulting from sexual arousal during a sexual activity, and to contemporaneously communicate characteristic information relating to the sensed series or pattern of electrical neural impulses to another device.

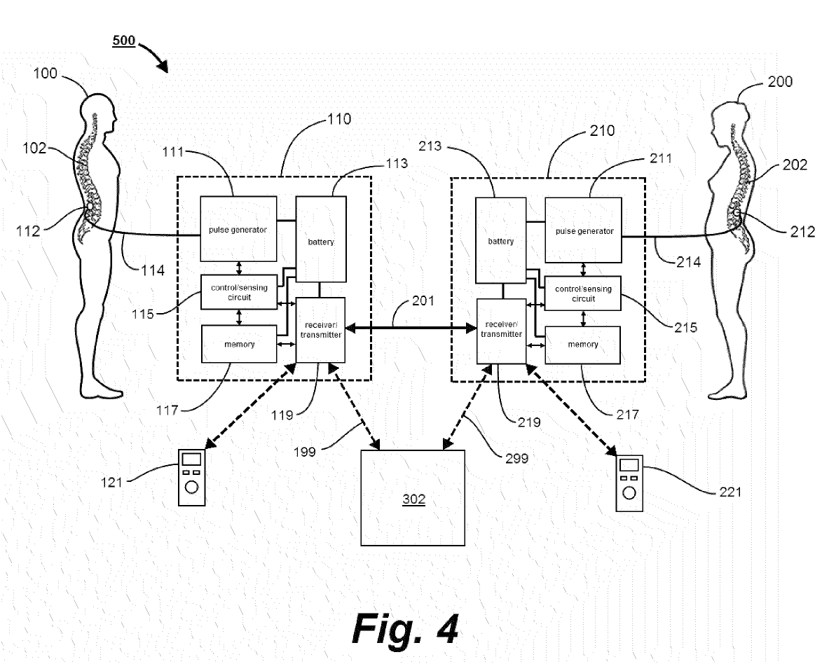

Details of one embodiment of a neural augmentation system 500 similar to that generally described above are shown schematically in FIG. 4. In this embodiment, the first and second IPGs 110, 210 can each include a pulse generator 111, 211, a battery 113, 213, a control processor 115, 215, memory 117, 217, and a wireless receiver/transmitter 119, 219. The batteries 113, 213 can be coupled to and power their associated components. Preferably, the batteries 113, 213 are rechargeable batteries, and include wireless recharging circuits. The first and second electrodes 112, 212 can be connected to their respective pulse generators 113, 213 by their corresponding leads 114, 214. Each IPG 110, 210 can be adapted to both apply and detect electrical impulses in a person's spinal cord. The internal control processors 115, 215 control operation of the IPGs 110, 210 and are coupled to their respective memories 117, 217 and receiver/transmitters 119, 219. Information such as data, software, and command signals can be received by the IPGs 110, 210 via their respective wireless receiver/transmitters 119, 219, and can be stored in the respective memories 117, 217 and/or used by the associated control processors 115, 215. Such information can be received from the other IPG 110, 210, another IPG (not shown), an associated external controller 121, 221, or another device 302, for example. The other device 302 can be a wireless relay/router or an external computer or processor, for example. The external controllers 121, 221 can be used to turn their associated IPG 110 or 210 on and off, for example, or to transmit specific commands to their associated IPG 110 or 210.

In the embodiment shown in FIG. 4, information can be communicated between the IPGs 110, 210 via a direct communication link 201 or via an intermediate device 302 and indirect communication links 199 and 299. Accordingly, the first IPG 110 can be implanted in the first person 100, can detect a series of electrical neural impulses in the first person's spine 102 and communicate the detected series of electrical impulses to the second IPG 210, can receive and process information from the second IPG 210, and can generate and apply another series of electrical impulses to the first person's spine 102 based upon information received from the second IPG 210. Similarly, the second IPG 210 can be implanted in the second person 200, can detect a series of electrical neural impulses in the second person's spine 202 and communicate the detected series of electrical impulses to the first IPG 110, can receive and process information from the first IPG 110, and can generate and apply another series of electrical impulses to the second person's spine 202 based upon the information received from the first IPG 110. In one embodiment, each IPG 110, 210 can substantially reproduce a pattern of electrical impulses detected and communicated by the other IPG 210, 110.

Alternatively, the IPGs 110, 210 can generate a series of electrical impulses that includes one or more modifications to a pattern of electrical impulses detected and communicated by the other IPG 210, 110. As discussed above, internal control processors 115, 215 control operation of the IPGs 110, 210 and are coupled to their respective memories 117, 217. One or both of the control processors 115, 215 can be programmed to cause their associated pulse generator 111 or 112 to generate a particular pattern of electrical impulses in response to receipt of certain commands or information from the other IPG 210, 110 or another device. For example, the first IPG 110 can be programmed to produce an enhanced pattern of electrical impulses when a series of electrical impulses communicated from the second IPG 210 includes certain characteristics. In one embodiment, the first IPG 110 is programmed to generate a particular pattern of electrical impulses that will stimulate the first person 100 to or near orgasm when the first IPG 110 detects that the second person 200 is experiencing or nearing orgasm based on information transmitted from the second IPG 210 to the first IPG 110. Similarly, the second IPG 210 can be programmed to generate a pattern of electrical impulses that will stimulate the second person 200 to or near orgasm when the second IPG 210 detects that the first person 100 is having or nearing orgasm based on information transmitted from the first IPG 110 to the first IPG 210. Alternatively, an intermediate device 302 like that shown in FIG. 4 can include a processor which is programmed to command either the first IPG 110 or second IPG 210 to generate a particular pattern of electrical impulses when the intermediate device 302 receives certain information from the other IPG 210, 110. For example, the intermediate device 302 may be a desktop or laptop computer programmed to command the first or second IPG 110, 210 to generate a particular pattern of electrical impulses in response to receipt of information communicated to the intermediate device by the other IPG 210 or 110. Information indicating that the first person 100 or second person 200 is experiencing or nearing orgasm, for example, may include certain characteristics of a detected electrical neural response in the other person 200, 100, such as a particular frequency, amplitude, or duration of electrical neural impulses detected in the other person's spinal cord 102, 202, or a particular combination of frequency, amplitude, and/or duration of the detected electrical neural impulses. As a result, both persons 100, 200 can substantially contemporaneously experience a heightened sense of arousal or orgasm during a sexual activity.

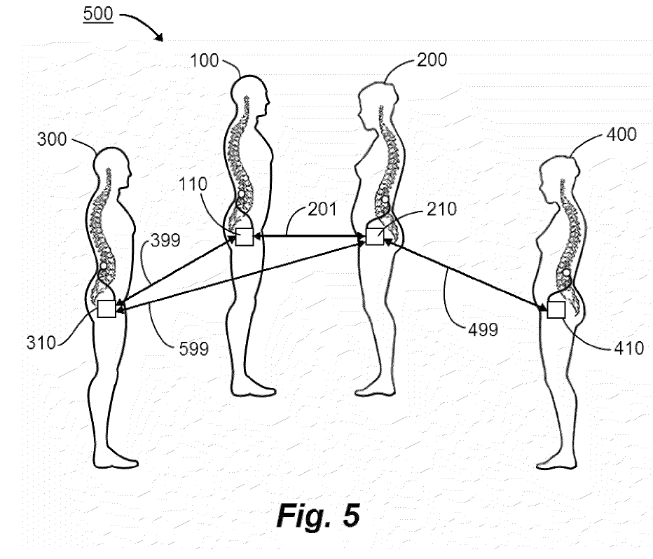

In the above descriptions, the sexual activities involved only two persons 100, 200. As shown in FIG. 5, however, a system 600 and method like that described above can also involve more than two persons and more than two IPGs. In this embodiment, a first IPG 110 is implanted in a first person 100, a second IPG 210 is implanted in a second person 200, and a third IPG 310 is implanted in a third person 300. Optionally, a fourth IPG 410 can be implanted in a fourth person 400. In this arrangement, each of the IPGs 110, 210, 310 and 410 is adapted to communicate with at least one of the other IPGs 110, 210, 310 and 410. For example, the first and second IPGs 110, 210 can be adapted to communicate with each other via a wireless communication link 201. Similarly, the third IPG 310 can be adapted to communicate with the second IPG 210 via wireless communication link 599, and/or to communicate with the first IPG 110 via wireless communication link 399. In this way, when the three persons 100, 200, 300 engage in a sexual activity, the sexual function of each person 100, 200, 300 can be augmented by electrical impulses generated by their respective IPG 110, 210 or 310 in response to a neural response detected by one or both of the other IPGs 110, 210 or 310. For example, when the first person 100, second person 200, and third person 300 engage in a sexual activity, the sexual function of the first person 100 can be neurally augmented by electrical impulses generated by the first IPG 110 in response to an electrical neural response detected by the second IPG 210 in the second person 200, and/or in response to an electrical neural response detected by the third IPG 310 in the third person 300. Similarly, the sexual function of the second person 200 can be neurally augmented by electrical impulses generated by the second IPG 210 in response to an electrical neural response detected by the first IPG 110 in the first person 100, and/or in response to an electrical neural response detected by the third IPG in the third person 300. Also similarly, the sexual function of the third person 300 can be neurally augmented by electrical impulses generated by the third IPG 310 in response to an electrical neural response detected by the first IPG 110 in the first person 100, and/or in response to an electrical neural response detected by the second IPG 210 in the second person 200. In all of the above cases, the pattern of electrical impulses generated by each IPG 110, 210, 310 can substantially duplicate or mimic a pattern of electrical neural impulses detected in another person 110, 210, 310, or can be a different pattern of electrical impulses produced in response to a particular pattern of electrical neural impulses detected in a partner 110, 210, 310.

As shown in FIG. 5, a fourth IPG 410 can be implanted in a fourth person 400, and can be adapted to wirelessly communicate with only the second IPG 210 via communication link 499, for example. In this arrangement, the sexual function of the fourth person 400 can be augmented by electrical impulses generated by the fourth IPG 410 in response to an electrical neural response detected by the second IPG 210 in the second person 200. In one embodiment, the fourth person 400 can observe a sexual activity between the second person 200 and the first person 100 and/or the third person 300, and can experience neural sensations being experienced by the second person 200 during such sexual activity. Alternatively, the fourth person 400 can actively participate in the sexual activity with the other persons 100, 200, 300. Though the above embodiments describe systems and methods for neurally augmenting the sexual functions of one to four persons during sexual activity involving two to four persons, the total numbers of persons and IPGs involved in a particular sexual activity and the various possible combinations of communication links between the involved IPGs are substantially unlimited.

The devices can be set with discrete digital codes if desired. This allows user's discretion as to whom is receiving transmitted information. This is analgous to having discrete codes on other devices, such as garage door openers. Only the person intended can receive the transmission.

Referring again to FIG. 4, in order for two IPGs 110, 210 to wirelessly communicate with each other, the first person 100 and second person 200 should be in close proximity to one another to ensure a satisfactory wireless connection. For example, the first person 100 and second person may need to be within about six feet of each other, based on the limitations of current equipment. It might become possible in the future to transmit the information over long distances via the internet, for example. In order to facilitate such communication between two IPGs 100, 200 or to increase the acceptable distance between the two persons 100, 200, the intermediate device 302 may include an antenna for relaying wireless signals between the IPGs 110, 210. For example, an antenna 302 could be placed on or within a bed or pillow being used for sexual activity by the first and second persons 100, 200, on or in a nearby article of furniture, or on or in another nearby article.

The above descriptions of various embodiments of the invention are intended to illustrate and highlight various aspects of the invention, and not to limit the invention to the specifically described embodiments. Persons of ordinary skill in the art will recognize that certain changes or modifications can be made to the described embodiments without departing from the scope of the invention. For example, though the pulse sensing and pulse generating devices are preferably implanted within participants in a sexual activity, it is possible for the implanted spinal cord electrodes to be connected to an external pulse generating and/or pulse sensing device by wires or the like. In addition, its possible for multiple implanted electrodes to be connected to a single external pulse generating and/or pulse sensing device by wires or the like. All such changes and modifications are intended to be within the scope of the appended claims.

What is claimed is:

1. A method of neurally augmenting sexual function, the method comprising:
(a) implanting at least one first electrode in a first person proximate to a selected portion of the first person's spinal cord and coupling the first electrode to a first electrical pulse sensor/generator;
(b) implanting at least one second electrode in a second person proximate to a selected portion of the second person's spinal cord and coupling the second electrode to a second electrical pulse sensor/generator;
(c) detecting a first series of electrical impulses with the first electrode and the first electrical pulse sensor/generator as the first person and second person engage in sexual activity, wherein the first series of electrical impulses is associated with neural activity occurring in the first person as a result of the sexual activity; and
(d) generating a second series of electrical impulses with the second electrical pulse sensor/generator and applying the second series of electrical impulses to the selected portion of the second person's spinal cord with the second electrode, wherein the second series of electrical impulses substantially corresponds to the first series of electrical impulses;
(e) whereby the second series of electrical impulses causes the second person to at least partially neurally sense the neural activity in the first person.

2. A method according to claim 1 wherein the second series of electrical impulses substantially mimics the first series of electrical nerve impulses.

3. A method according to claim 1 wherein the selected portion of the first person's spinal cord and the selected portion of the second person's spinal cord correspond to substantially the same portion of a human spinal cord.

4. A method according to claim 1 wherein selected portions of the first and second person's spinal cords are T8-S3.

5. A method according to claim 1 wherein applying the second series of electrical impulses to the selected portion of the second person's spinal cord is substantially contemporaneous with detecting the first series of electrical nerve impulses.

6. A method according to claim 1 wherein the sexual activity comprises sexual intercourse.

7. A method according to claim 1 further comprising:
(a) implanting the first electrical pulse sensor/generator in the first person, wherein the first electrical pulse sensor/generator comprises a transmitter; and
(b) implanting the second electrical pulse sensor/generator in the second person, wherein the second electrical pulse sensor/generator comprises a receiver;
(c) communicating the first series of electrical impulses from the transmitter to the receiver; and
(d) generating the second series of electrical impulses with the second electrical pulse sensor/generator in response to communicating the first series of electrical nerve impulses from the transmitter to the receiver.

8. A method according to claim 7 wherein communicating the first series of electrical nerve impulses from the transmitter to the receiver comprises a wireless transmission.

9. A method according to claim 7 wherein the first electrical pulse sensor/generator is coupled to the first electrode by a first implanted lead, and the second electrical pulse sensor/generator is coupled to the second electrode by a second implanted lead.

10. A method according to claim 1 further comprising:
(a) detecting a third series of electrical impulses with the second electrode and the second electrical pulse sensor/generator as the first person and second person engage in the sexual activity, wherein the third series of electrical impulses is associated with neural activity occurring in the second person as a result of the sexual activity; and
(b) generating a fourth series of electrical impulses with the first electrical pulse sensor/generator and applying the fourth series of electrical impulses to the selected portion of the first person's spinal cord with the first electrode, wherein the fourth series of electrical impulses substantially corresponds to the third series of electrical impulses;
(c) whereby the fourth series of electrical impulses causes the first person to at least partially neurally sense the neural activity in the second person.

11. A method according to claim 10 wherein transmitting the fourth series of electrical impulses is substantially contemporaneous with detecting the third series of electrical nerve impulses.

12. A method according to claim 10 wherein detecting the first and third series of electrical impulses and transmitting the second and fourth series of electrical impulses are substantially contemporaneous.

13. A method according to claim 10 further comprising:
(a) implanting the first electrical pulse sensor/generator, wherein the first electrical pulse sensor/generator comprises a receiver; and
(b) implanting the second electrical pulse sensor/generator in the second person, wherein the second electrical pulse sensor/generator comprises a transmitter;
(c) communicating the third series of electrical nerve impulses from the transmitter to the receiver; and
(d) generating the fourth series of electrical impulses with the first electrical pulse sensor/generator in response to communicating the third series of electrical nerve impulses from the transmitter to the receiver.

14. A method according to claim 13 wherein the first electrical pulse sensor/generator is coupled to the first electrode by a first implanted lead, and the second electrical pulse sensor/generator is coupled to the second electrode by a second implanted lead.

15. A method according to claim 1 wherein the sexual activity comprises an orgasm by the first person, and wherein the second series of electrical impulses is at least partially modified in response to the orgasm.

16. A method of augmenting sexual function comprising:
(a) detecting a first neural response in a first person during sexual activity with a second person; and
(b) artificially inducing a second neural response in the second person that substantially corresponds to the first neural response, wherein the second neural response is substantially contemporaneous with the first neural response.

17. A method according to claim 16 wherein detecting the first neural response in the first person comprises detecting an electrical neural response in a selected portion of the first person's spinal cord.

18. A method according to claim 16 wherein artificially producing a second neural response in the second person comprises applying a pattern of electrical pulses to a selected portion of the second person's spinal cord.

19. A method according to claim 16 wherein detecting the first neural response in the first person comprises detecting a pattern of electrical nerve impulses in a portion of the first person's spinal cord, and wherein artificially producing a second neural response in the second person comprises applying a series of electrical impulses to a portion of the first person's spinal cord, wherein the portion of the first person's spinal cord and the portion of the second person's spinal cord correspond to substantially the same portion of a human spinal cord.

20. A method according to claim 16 wherein detecting the first neural response in the first person comprises detecting a pattern of electrical nerve impulses in a portion of the first person's spinal cord, and wherein artificially producing a second neural response in the second person comprises applying a series of electrical impulses to a portion of the second person's spinal cord, wherein the applied series of electrical impulses substantially mimics the detected pattern of electrical nerve impulses.

21. A method according to claim 16 wherein detecting the pattern of electrical nerve impulses in the first person comprises detecting the pattern of electrical nerve impulses with at least one electrode implanted in the first person proximate to a selected portion of the first person's spinal cord.

22. A method according to claim 16 wherein artificially producing the second neural response in the second person comprises transmitting a series of electrical impulses to an electrode implanted in the second person proximate to a selected portion of the second person's spinal cord.

23. A method according to claim 16 further comprising:
(a) detecting a third neural response in the second person during the sexual activity with the first person; and
(b) artificially inducing a fourth neural response in the first person that substantially corresponds to the third neural response, wherein the fourth neural response is substantially contemporaneous with the third neural response.

24. A method according to claim 23 wherein detecting the third neural response in the second person comprises detecting electrical neural activity in a selected portion of the second person's spinal cord.

25. A method according to claim 23 wherein artificially producing a fourth neural response in the first person comprises applying a pattern of electrical pulses to a selected portion of the first person's spinal cord.

26. A method according to claim 23 wherein detecting the third neural response in the second person comprises detecting a pattern of electrical nerve impulses in a portion of the second person's spinal cord, and wherein artificially producing a fourth neural response in the first person comprises applying a series of electrical impulses to a portion of the first person's spinal cord, wherein the portion of the first person's spinal cord and the portion of the second person's spinal cord correspond to substantially the same portion of a human spinal cord.

27. A method according to claim 23 wherein detecting the third neural response in the second person comprises detecting a pattern of electrical nerve impulses in a portion of the second person's spinal cord, and wherein artificially producing a fourth neural response in the first person comprises applying a series of electrical impulses to a portion of the first person's spinal cord, wherein the applied series of electrical impulses substantially mimics the detected pattern of electrical nerve impulses.

28. A method according to claim 23 wherein detecting the pattern of electrical nerve impulses in the second person comprises detecting the pattern of electrical nerve impulses with at least one electrode implanted in the second person proximate to a selected portion of the second person's spinal cord.

29. A method according to claim 23 wherein artificially producing the fourth neural response in the first person comprises transmitting a series of electrical impulses to an electrode implanted in the first person proximate to a selected portion of the first person's spinal cord.

30. A method according to claim 16 further comprising:
(a) implanting a first electrical pulse sensor/generator in the first person; and
(b) implanting a second electrical pulse sensor/generator in the second person;
(c) detecting the first neural response with the first electrical pulse sensor/generator and communicating information related to the first neural response from the first electrical pulse sensor/generator to the second electrical pulse sensor/generator; and
(d) artificially producing the second neural response with the second electrical pulse sensor/generator in response to the information communicated from the first electrical pulse sensor/generator.

31. A method according to claim 30 wherein the first electrical pulse sensor/generator includes a wireless transmitter and the second electrical pulse sensor/generator includes a wireless receiver, and wherein the information is wirelessly transmitted from the transmitter to the receiver.

32. A method according to claim 30 further comprising:
(a) detecting a third neural response in the second person with the second electrical pulse sensor/generator during the sexual activity with the first person; and
(b) artificially producing a fourth neural response in the first person with the first electrical pulse sensor/generator that is substantially similar to the third neural response.

33. A method according to claim 30 wherein detecting the first and third neural responses and producing the second and fourth neural responses occur substantially contemporaneously.

34. A system for augmenting sexual function during sexual activity between a first person and a second person, the system comprising:
(a) a first implantable device adapted to be implanted in the first person and comprising a sensor and a transmitter; and
(b) a second implantable device adapted to be implanted in the second person and comprising an electrode, a current generator coupled to the electrode, and a receiver;
(c) wherein the first device is adapted to detect a series of electrical neural impulses in a portion of the first person's spinal cord with the sensor, and to transmit information related to the series of electrical neural impulses from the transmitter to the receiver; and
(d) wherein the second device is adapted to produce a pattern of electrical impulses in response to the information transmitted from the transmitter to the receiver and to convey the pattern of electrical impulses to a portion of the second person's spinal cord via the electrode;
(e) wherein the pattern of electrical impulses is substantially similar to the series of electrical neural impulses.
